(19) United States
(12) Patent Application Publication
(10) Pub. No.: US 2022/0340674 A1
(43) Pub. Date: Oct. 27, 2022

(54) METHOD FOR THE PRODUCTION OF BISPECIFIC FCYRIII X CD30 ANTIBODY CONSTRUCT

(71) Applicant: Affimed GmbH, Heidelberg (DE)

(72) Inventors: Claudia WALL, Heidelberg (DE); Michael WEICHEL, Heidelberg (DE)

(21) Appl. No.: 17/807,211

(22) Filed: Jun. 16, 2022

Related U.S. Application Data
(63) Continuation of application No. PCT/EP2020/087896, filed on Dec. 27, 2020.

(30) Foreign Application Priority Data
Dec. 27, 2019 (EP) 19219925.5

(51) Int. Cl. C07K 16/28

(52) U.S. Cl. CPC C07K 16/2878; C07K 16/283; C07K 2317/31; C07K 2317/565; C07K 2317/10

(57) ABSTRACT

The invention relates to a method for the production of a bispecific CD30×CD16A antibody construct comprising a first binding domain for FcγRIIIa comprising the steps chromatographically capturing the antibody construct from a solution; eluting the antibody construct from the capture matrix; reducing the pH in the solution of the eluted antibody construct to low pH, incubating the antibody construct under these conditions for at least 40 h and neutralizing thereafter.

This application is a continuation of PCT/EP2020/087896, filed Dec. 27, 2020; which claims priority to EP Application No. 19219925.5, filed Dec. 27, 2019. The contents of the above applications are incorporated herein by reference in their entirety.

### FIELD OF THE INVENTION

The present invention provides a method for the production of a bispecific FcγRIII×CD30 antibody construct and bispecific antibody constructs produced by said method.

### BACKGROUND OF THE INVENTION

A recovery and purification process for antibody production needs to remove impurities such as host cell protein, DNA, viruses, endotoxins and other species while an acceptable yield of active antibody is obtained.

Chromatography is a widely used separation and purification technology for antibodies. A number of chromatographic resins are applied for recovering and purification of antibodies such as, for example, Protein A affinity chromatography, ion exchange chromatography, anion exchange chromatography, hydrophobic interaction chromatography (HIC), hydrophobic charge induction chromatography (HCIC), ceramic hydroxyapatite chromatography or multimodal chromatography.

HCIC is a mixed mode chromatography. An HCIC resin contains a ligand, for example sorbent 4-Mercapto-Ethyl-Pyridin (MEP HyperCel™), that is ionizable and hydrophobic at physiologically neutral or slightly acidic pH (e.g. pH 6-9) for binding the antibody construct via non-specific hydrophobic interaction to the column. For elution the pH is reduced thereby disrupting the hydrophobic binding by electrostatic charge repulsion towards the eluate due to the pH shift.

Further, viral clearance steps by removing and/or inactivating viruses have to incorporated in a purification process for antibody production from mammalian cells. For example, viral clearance can be achieved by a low pH hold of the chromatography eluate. For example, a low pH hold following a chromatography step can be accomplished by decreasing the pH of the eluate to about 3.4 to 3.6 followed by neutralizing the eluate by increasing the pH to about 7. A pH<3.6 has been reported as robust in achieving retrovirus inactivation (Qi Chen, PDA J Pharm Sci and Tech, 2014, 68, 17-22). Typically, a low pH hold is performed for about 30 minutes to about 60 minutes.

### BRIEF DESCRIPTION OF THE DRAWING

FIG. 1: Kinetic assay of acidic MEP eluate incubation of a CD30×CD16A bispecific antibody

### DEFINITIONS

The term “antibody construct” refers to a molecule or class of molecules in which the structure and/or function is/are based on the structure and/or function of an antibody. Examples for such an antibody include e.g. full-length or whole immunoglobulin molecules and/or constructs drawn from the variable heavy chain (VH) and/or variable light chain (VL) domains of an antibody or fragment thereof. An antibody construct is hence capable of binding to its specific target or antigen. Furthermore, the binding domain of an antibody construct defined in the context of the invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs, respectively all three heavy chain CDRs of a single-domain antibody (sdAb) derived construct. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs defined in the context of the invention are based include for example monoclonal, recombinant, chimeric, deimmunized, humanized and human antibodies.

The binding domain of an antibody construct defined in the context of the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library).

Also, within the definition of “binding domain” or “domain which binds” are fragments of full-length antibodies, such as VH, VHH, VL, (s)dAb, Fv, Fd, Fab, Fab′, F(ab′)₂ or “r IgG” (“half antibody”). Antibody constructs as defined in the context of the invention may also comprise modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Plueckthun in The Pharmacology of Monoclonal Antibodies, vol. 1 13, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

Furthermore, the definition of the term “antibody construct” includes monovalent, bivalent and polyvalent/multivalent constructs and, thus, bispecific constructs, specifically binding to only two antigenic structure, as well as polyspecific/multispecific constructs, which specifically bind more than two antigenic structures, e.g. three, four or more, through distinct binding domains. Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010, Little, Recombinant Antibodies for Immunotherapy, Cambridge University Press 2009 and Nevoltris and Chames, Antibody Engineering—Methods and Protocols, Springer 2018.

The term “valent” denotes the presence of a determined number of antigen-binding domains in the antigen-binding protein. A natural IgG has two antigen-binding domains and is bivalent. The antigen-binding proteins as defined in the context of the invention are at least trivalent. Examples of tetra-, penta- and hexavalent antigen-binding proteins are described herein.

The term “bispecific” as used herein refers to an antibody construct which is “at least bispecific”, i.e., it comprises at least a first binding domain and a second binding domain, wherein the first binding domain binds to one antigen or target (here: NK cell receptor, e.g. CD16a), and the second binding domain binds to another antigen or target (here: the target cell surface antigen CD30). Accordingly, antibody constructs as defined in the context of the invention comprise specificities for at least two different antigens or targets. For example, the first domain does preferably bind to an extracellular epitope of an NK cell receptor of one or more of the species selected from human, Macaca spec. and rodent species.

The term “NK cell receptor” as used in the context of the invention defines proteins and protein complexes on the surface of NK cells. Thus, the term defines cell surface molecules, which are characteristic to NK cells, but are not necessary exclusively expressed on the surface of NK cells but also on other cells such as macrophages or T cells. Examples for NK cell receptors comprise, but are not limited to FcγRIII (CD16a, CD16b), NKp46 and NKG2D.

“CD16a” refers to the activating receptor CD16a, also known as FcγRIIIA, expressed on the cell surface of NK cells. CD16a is an activating receptor triggering the cytotoxic activity of NK cells. The affinity of antibodies for CD16a directly correlates with their ability to trigger NK cell activation, thus higher affinity towards CD16a reduces the antibody dose required for activation. The antigen-binding site of the antigen-binding protein binds to CD16a, but not to CD16b. For example, an antigen-binding site comprising heavy (VH) and light (VL) chain variable domains binding to CD16a, but not binding to CD16B, may be provided by an antigen-binding site which specifically binds to an epitope of CD16a which comprises amino acid residues of the C-terminal sequence SFFPPGYQ (SEQ ID NO:18) and/or residues G130 and/or Y141 of CD16a (SEQ ID NO: 19)) which are not present in CD16b.

“CD16b” refers to receptor CD16b, also known as FcγRIIIB, expressed on neutrophils and eosinophils. The receptor is glycosylphosphatidyl inositol (GPI) anchored and is understood to not trigger any kind of cytotoxic activity of CD16b positives immune cells.

The term “target cell surface antigen” refers to an antigenic structure expressed by a cell and which is present at the cell surface such that it is accessible for an antibody construct as described herein. The “target cell surface antigen”, to which the bispecific antibody constructs described herein bind to is CD30. CD30 also known as TNFRSF8, is a cell membrane protein of the tumor necrosis factor receptor family and tumor marker.

The term “bispecific antibody construct” as defined in the context of the invention also encompasses multispecific antibody constructs such as trispecific antibody constructs, the latter ones including three binding domains, or constructs having more than three (e.g. four, five . . . ) specificities. Examples for bi- or multispcific antibody constructs are provided e.g. in WO 2006/125668, WO 2015/158636, WO 2017/064221, WO 2019/175368, WO 2019/198051 and Ellwanger et a. (MAbs. 2019 July; 11(5):899-918).

Given that the antibody constructs as defined in the context of the invention are (at least) bispecific, they do not occur naturally and they are markedly different from naturally occurring products. A “bispecific” antibody construct or immunoglobulin is hence an artificial hybrid antibody or immunoglobulin having at least two distinct binding sides with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains (VH/VL) of the antibody construct of the present invention may or may not comprise peptide linkers (spacer or connector peptides). The term “peptide linker” comprises in accordance with the present invention an amino acid sequence by which the amino acid sequences of one (variable and/or binding) domain and another (variable and/or binding) domain of the antibody construct defined herein are linked with each other. The peptide linkers can also be used to fuse the third domain to the other domains or an Fc part of the antibody construct defined herein. An essential technical feature of such peptide linker is that it does not comprise any polymerization activity.

The antibody constructs as defined in the context of the invention are preferably “in vitro generated antibody constructs”. This term refers to an antibody construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations, deaminations, oxidation and glycosylations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target cell surface antigen, (Schier, Human Antibodies Hybridomas 7 (1996), 97-105; Malmborg, J. Immunol. Methods 183 (1995), 7-13). Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et ai, Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE®, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit., Little (2009), loc. cit. and Nevoltris and Chames (2018), loc. cit.

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human target cell surface antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “prioritized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs, variants or fragments thereof (such as Fv, Fab, Fab′, F(ab′)₂ or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor ei a/., Immunology Today, 4: 7279, 1983; Olsson et al., Meth. Enzymol., 92: 3-16, 1982, and EP 239 400).

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains as defined in the context of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antibody constructs defined herein are “isolated” or “substantially pure” antibody constructs. “Isolated” or “substantially pure”, when used to describe the antibody constructs disclosed herein, means an antibody construct that has been identified, separated and/or recovered from a component of its production environment. The antibody construct is obtained from a solution comprising the antibody construct and one or more other components, i.e. impurities. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art.

In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

The antibody construct is isolated from a “solution” by the at least one chromatographic capture step as a purification step. Such “solution” is a mixture comprising the antibody construct and one or more impurities as other components. The solution can be directly obtained from the host cell or microorganism producing the antibody construct, for example, cell culture supernatant or harvested cell culture fluid. The solution can be obtained by physically separating cells from the antibody construct and other other components and, optionally, conditioning by a buffer and/or dilution before subjecting it to a chromatographic capture step.

The term “binding domain” characterizes in connection with the present invention a domain which (specifically) binds to/interacts with/recognizes a given target epitope or a given target side on the target molecules (antigens), e.g. a NK cell receptor antigen, e.g. CD16, and the target cell surface antigen CD30, respectively. The structure and function of the first binding domain (recognizing e.g. CD16), and preferably also the structure and/or function of the second binding domain (recognizing the target cell surface antigen), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is affected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domain which binds to the NK cell receptor antigen, e.g. CD16 and/or the binding domain which binds to the target cell surface antigen CD30 is/are human, humanized or murine derived chimeric binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a human binding domain against the target cell surface antigen and a human binding domain against CD16 in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), here: the NK cell receptor, e.g. CD16a, and the target cell surface antigen, respectively.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or the region comprising the epitope implies that a binding domain exhibits appreciable affinity for the epitope/the region comprising the epitope on a particular protein or antigen (here: the NK cell receptor, e.g. CD16a, and the target cell surface antigen, respectively) and, generally, does not exhibit significant reactivity with proteins or antigens other than the NK cell receptor, e.g. CD16a, and the target cell surface antigen CD30. “Appreciable affinity” includes binding with an affinity of about 10⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the NK cell receptor, e.g. CD16a, and the target cell surface antigen. Preferably, a binding domain as defined in the context of the invention does not essentially or substantially bind to proteins or antigens other than the NK cell receptor, e.g. CD16a, and the target cell surface antigen (i.e., the first binding domain is not capable of binding to proteins other than the NK cell receptor, e.g. CD16a, and the second binding domain is not capable of binding to proteins other than the target cell surface antigen).

The term “does not essentially/substantially bind” or “is not capable of binding” means that a binding domain of the present invention does not bind a protein or antigen other than the NK cell receptor, e.g. CD16a, and the target cell surface antigen, i.e., does not show reactivity of more than 30%, preferably not more than 20%, more preferably not more than 10%, particularly preferably not more than 9%, 8%, 7%, 6% or 5% with proteins or antigens other than the NK cell receptor, e.g. CD16a, and the target cell surface antigen, whereby binding to the NK cell receptor, e.g. CD16a, and the target cell surface antigen, respectively, is set to be 100%.

Specific binding is believed to be affected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding side.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit; Chothia et al., J. Mol. Biol, 1987, 196: 901-917; and MacCallum et al., J. Mol. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. A global reference in immunoinformatics is the three-dimensional (3D) structure database of IMGT (international ImMunoGenetics information system) (Ehrenmann et al., 2010, Nucleic Acids Res., 38, D301-307). The IMGT/3Dstructure-DB structural data are extracted from the Protein Data Bank (PDB) and annotated according to the IMGT concepts of classification, using internal tools. Thus, IMGT/3Dstructure-DB provides the closest genes and alleles that are expressed in the amino acid sequences of the 3D structures, by aligning these sequences with the IMGT domain reference directory. This directory contains, for the antigen receptors, amino acid sequences of the domains encoded by the constant genes and the translation of the germline variable and joining genes. The CDR regions of our amino acid sequences were preferably determined by using the IMGT/3Dstructure database.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The antibody construct defined in the context of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antibody constructs characterized by the identified CDRs may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag (HHHHHH) (SEQ ID NO:20) is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the antibody constructs described herein are also contemplated. For example, it may be desirable to improve the binding affinity and/or other biological properties of the antibody construct. Amino acid sequence variants of the antibody constructs are prepared by introducing appropriate nucleotide changes into the antibody constructs nucleic acid, or by peptide synthesis. All of the below described amino acid sequence modifications should result in an antibody construct which still retains the desired biological activity (binding to the NK cell receptor, e.g. CD16a, and the target cell surface antigen) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, Ile, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gin, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within a third domain of the antibody construct defined in the context of the invention. An insertional variant of the antibody construct defined in the context of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as Arg, Asp, His, Lys, and Glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as the NK cell receptor, e.g. CD16a, and the target cell surface antigen binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody construct may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antibody construct retains its capability to bind to the NK cell receptor, e.g. CD16a via the first domain and to the target cell surface antigen via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| | | Preferred |
|---|---|---|
| Original | Exemplary Substitutions | Substitutions |
| Ala (A) | val, leu, ile | val |
| Arg (R) | lys, gln, asn | lys |
| Asn (N) | gln, his, asp, lys, arg | gln |
| Asp (D) | glu, asn | glu |
| Cys (C) | ser, ala | ser |
| Gln (Q) | asn, glu | asn |
| Glu (E) | asp, gln | asp |
| Gly (G) | ala | ala |
| His (H) | asn, gln, lys, arg | arg |
| Ile (I) | leu, val, met, ala, phe | leu |
| Leu (L) | norleucine, ile, val, met, ala | lie |
| Lys (K) | arg, gln, asn | arg |
| Met (M) | leu, phe, ile | leu |
| Phe (F) | leu, val, ile, ala, tyr | tyr |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gin; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL defined in the context of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

Certain embodiments provide pharmaceutical compositions comprising the antibody construct defined in the context of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of one aspect of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18” Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine
antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH; examples of buffers are borate, bicarbonate,
Tris-HCl, citrates, phosphates or other organic acids, succinate, phosphate, and histidine; for example, Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetra acetic acid (EDTA);
isotonic and absorption delaying agents;
complexing agents such as caffeine, polyvinylpyrrolidone, beta-cyclodextrin or hydroxypropyl-beta-cyclodextrin) fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);
sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### DETAILED DESCRIPTION OF THE INVENTION

A characteristic of down stream procedures for antibodies is a capture step of the protein from the cell culture in order to reduce the very large volume prior to the following purification steps. Such capture step can be a chromatographic procedure.

The present invention is based on the unexpected finding that a high yield of active antibody can be recovered despite the harsh conditions by accomplishing an extended treatment of a chromatographic eluate at a low pH.

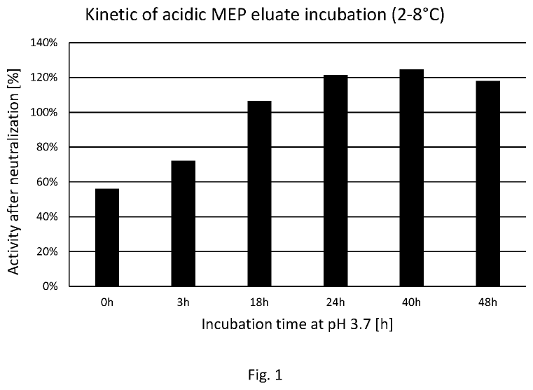

FIG. 1 shows that a hydrophobic charge induction chromatography (HCIC) using as sorbent 4-Mercapto-Ethyl-Pyridin (MEP HyperCel™) was able to capture a CD30×CD16A bispecific antibody from the cell culture but only about 50% of the antibody activity was recovered after eluting at pH 3.7 and neutralizing it to pH7.0 (0 h). Surprisingly, the present inventors found that 100% antibody activity regained after an about two days incubation at pH3.7. This is in contrast to an expected protein deterioration under acidic conditions. The kinetics of this phenomenon is shown in FIG. 1.

Further, the low pH treatment revealed a reduction of high molecular weight forms as described in Example 2, concluding a benefit for the purification of CD30×CD16A bispecific antibody.

Incubation at low pH has the additional advantage of reducing the virus titer. However, such acid incubation steps in down stream processing of the prior art are kept to about one hour for avoiding protein deterioration.

Thus, in a first aspect the present invention provides a method for the production of bispecific antibody construct comprising a first binding domain for FcγRIII and a second binding domain for CD30, the method comprising the following steps
(a) chromatographically capturing the antibody construct from a solution;
(b) eluting the antibody construct from the capture matrix;
(c) reducing the pH in the solution of the eluted antibody construct to low pH in a range of 2.5 pH to 3.9 pH and incubating the antibody construct under these conditions for at least 40h;
(d) neutralizing to a pH in the range of pH 4.5 to pH 8.0.

As known in the art, chromatography is a laboratory technique for the separation of a mixture. The mixture is dissolved in a fluid called the mobile phase, which carries it through a structure holding another material called the stationary phase. Chromatographic methods are broadly used in the field of antibody technology; see Gottschalk (editor), Process Scale Purification of Antibodies 2009.

As described herein below in detail, the reduction of the pH in the solution as described for step (c) of the method of the invention is achieved by adding an acid solution. In line with the method of the invention it is preferred that the incubation according to step (c) is performed at a temperature of ≤12° C., preferable ≤10° C. More preferably, the incubation according to step (c) is performed at a temperature in a range of 2° C. to 10° C. and most preferably in a range of 2° C. to 8° C.

The incubation of the antibody construct in the low pH solution as set forth in step (c) of the method of the invention is set forth for at least 40h and stopped by neutralization of the solution according to step (d) of the method of the invention. The neutralized solution is adapted to a pH in the range of pH 4.5 to pH 8.0 by addition of a basic (alkaline) solution. It is preferred that the neutralized solution is adapted to a pH in the range of pH 6.5 to pH 7.5. More preferably, the neutralized solution is adapted to a pH in the range of pH 6.8 to pH 7.2.

As described herein above, the incubation of an antibody construct, which is a protein, in low pH (acid) conditions is stressful for any protein. Nevertheless, for a short period of time (commonly in the range of 5 minute and 120 minutes; see Chen 2015, PDA J Pharm Sci and Tech, 68, 17, Gottschalk (editor), Process Scale Purification of Antibodies 2009 Wiley chapter 4.5.2) such low pH step is part of a standard procedure in the downstream processing of biologics for the inactivation of possible virus contamination.

In line with the method of the invention the first binding domain of the antibody construct for FcγRIII binds to CD16A.

It is preferred for the method of the invention that the antibody construct comprises at least four variable domains from the group consisting of
(a) a heavy chain variable domain specific for CD16A (VH_CD16A) comprising a heavy chain CDR1 having the amino acid sequence set forth in SEQ ID NO:1, a heavy chain CDR2 having the amino acid sequence set forth in SEQ ID NO:2, a heavy chain CDR3 having the amino acid sequence set forth in SEQ ID NO: 3;
(b) a light chain variable domain specific for CD16A (VL_CD16A) comprising a light chain CDR1 having an amino acid sequence set forth in SEQ ID NO:4, a light chain CDR2 having an amino acid sequence set forth in SEQ ID NO: 5, and a light chain CDR3 having an amino acid sequence set forth in SEQ ID NO: 6;
(c) heavy chain variable domain specific for CD30 (VH_CD30A) comprising a heavy chain CDR1 having the amino acid sequence set forth in SEQ ID NO:7, a heavy chain CDR2 having the amino acid sequence set forth in SEQ ID NO:8, a heavy chain CDR3 having the amino acid sequence set forth in SEQ ID NO: 9;
(d) a light chain variable domain specific for CD30A (VL_CD30A) comprising a light chain CDR1 having an amino acid sequence set forth in SEQ ID NO:10, a light chain CDR2 having an amino acid sequence set forth in SEQ ID NO: 11, and a light chain CDR3 having an amino acid sequence set forth in SEQ ID NO: 12.

It is also preferred for the method of the invention that the variable domains of the antibody construct are linked one after another by peptide linkers L1, L2 and L3 consisting of 12 or less amino acid residues and positioned within each of the two polypeptide chains from the N-terminus to the C-terminus in the order: VH_CD30-L1-VL_CD16A-L2-VH_CD16A-L3-VL_CD30.

In a preferred embodiment of the method of the invention the linker L2 of the antibody construct consists of 3 to 9 amino acid residues.

Moreover, it is preferred for the method of the invention that the antibody construct comprises an amino acid sequence as set forth in SEQ ID NO:13.

In a preferred embodiment of the method of the invention the pH in step (c) is in the range of pH 3.0 to pH 3.8, preferably in the range of pH 3.5 to pH 3.75, more preferably in the range of pH 3.65 to pH 3.7.

It is also preferred for the method of the invention that the antibody construct is incubated in step (c) for at least 48h, preferably for at least 96h, in the low pH. As described herein above, the incubation according to step (c) is performed at a temperature of ≤12° C., preferable ≤10° C. More preferably, the incubation according to step (c) is performed at a temperature in a range of 2° C. to 10° C. and most preferably in a range of 2° C. to 8° C.

In a further embodiment, the antibody construct is incubated in step (c) at room temperature (RT) as used for storage of pharmaceuticals (e.g. defined by United States Pharmacopeia or European Pharmacopeia), for example between 15 and 30° C., particularly 15 to 25° C., most particularly 20 to 25° C.

In certain embodiments, step (c) comprises at least one week, at least 3 weeks, 3 to 5 weeks or up to 5 weeks in the low pH, before step (d) is performed.

In further embodiments, the antibody is incubated in step (c) at room temperature for at least one week, at least 3 weeks, 3 to 5 weeks or up to 5 weeks in the low pH.

In a preferred embodiment of the method of the invention the chromatographic capturing method in step (a) is selected form the group consisting of a protein L chromatography, an anion exchange chromatography (AEX), a cation exchange chromatography (CEX), a hydrophobic interaction chromatography (HIC) or a mixed mode chromatography (MMC).

Mixed-mode chromatography (MMC), or multimodal chromatography, refers to chromatographic methods that utilize more than one form of interaction between the stationary phase and analytes in order to achieve their separation. Accordingly, this type of chromatographic method is commonly applied of the preparation and isolation of biologics, e.g. antibodies and antibody constructs. MMC can be classified into physical MMC and chemical MMC. In the former method, the stationary phase is constructed of two or more types of packing materials. In the chemical method, just one type of packing material containing two or more functionalities is used. Examples for chemical methods comprise ion exchange chromatography (IEC) plus hydrophobic interaction chromatography (HIC), ICE plus reversed phase liquid chromatography (RPLC), Hydrophilic interaction chromatography or hydrophilic interaction liquid chromatography (HILIC) plus RPLC, HILIC plus IEC and seize exclusion chromatography (SEC) plus IEC.

It is preferred for the method of the invention that the chromatographic capturing in step (a) is either a protein L chromatography, preferably using TOYOPEARL® AF-rProtein L-650F, or Capto L from GE, or hydrophobic charge induction chromatography (HCIC).

In a preferred embodiment of the method of the invention the hydrophobic charge induction chromatography (HCIC) is a Mixed-Mode Chromatography Sorbent (e.g. MEP Hypercel™). It is also preferred that the HCIC is followed by an anion exchange chromatography (AEX) and/or a cation exchange chromatography (CEX).

It is preferred for the method of the invention that the method further comprises the additional steps:
(e) capturing the antibody construct from the solution by at least one chromatographic method selected from the group consisting of an anion exchange chromatography (AEX), a cation exchange chromatography (CEX), a hydrophobic interaction chromatography (HIC) or a mixed mode chromatography (MMC).

It is preferred for the method of the invention that the chromatographic capturing in step (e) is either a protein L chromatography, preferably using TOYOPEARL® AF-rProtein L-650F, or hydrophobic charge induction chromatography (HCIC). In a preferred embodiment of the method of the invention the hydrophobic charge induction chromatography (HCIC) is a Mixed-Mode Chromatography Sorbent (e.g. MEP Hypercel™). It is also preferred that the HCIC is followed by an anion exchange chromatography (AEX) and/or a cation exchange chromatography (CEX).

In a further embodiment the method of the invention comprises an additional chromatographic capture step before step (a). Such additional chromatographic capture step may be, for example, an anion exchange chromatogtaphy. In a certain embodiment the method of the invention comprises a chromatographic capture step, for example an anion exchange chromatography, followed downstream by step (a) as described above and the chromatographic method of step (e).

In a further embodiment the method of the invention comprises at least one additional filtration step. Such a filtrations step may be after step (d). For example, the filtration step may be between step (d) and step (e). In a particular embodiment the method of the invention may comprise a further additional filtration step after step (e) and/or before step (a). For example, the method of the invention may comprise filtration steps before step (a), between step (d) and step (e), and after step (e). Preferably, such filtration step is an ultrafiltration.

Moreover, it is preferred for the method of the invention that the elution of the antibody construct in step (b) is performed using a buffer selected from the group consisting of buffers comprising sodium acetate/acetic acid, sodium formate/formic acid, sodium citrate/citric acid, and sodium succinate/succinic acid. The respective buffers are used in concentration ranges of approximately 10 mM up to approximately 100 mM in rare cases up to 200 mM depending on the applied parameters.

For the neutralization according to step (d) of the method of the invention it is preferred that such neutralization is achieved by adding a buffer or solution of higher pH. Examples for suitable buffer or solutions include but are not limited to Tris buffered solutions like e.g. an AEX buffer 20 mM Tris-HCL; pH7.0. Of course, the person skilled in the art is aware of suitable alternative buffer solutions for the neutralization according to step (d).

It is further preferred for the method of the invention that the antibody construct is formulated as a pharmaceutical composition in a step (f).

The present invention also provides an antibody construct produced by a method of the invention.

In one embodiment of the pharmaceutical composition according to one aspect of the invention the composition is administered to a patient intravenously.

Methods and protocols for the intravenous (iv) administration of pharmaceutical compositions described herein are well known in the art.

In one aspect of the invention a pharmaceutical composition is provided said pharmaceutical composition is used in the prevention, treatment or amelioration of a CD30⁺ proliferative disease or a tumorous disease. Preferably, said tumorous disease is a malignant disease, preferably cancer.

In one embodiment of the pharmaceutical composition of the invention the identified malignant disease is selected from the group consisting of Hodgkin lymphoma, Non-Hodgkin lymphoma, leukemia, multiple myeloma and solid tumors.

Also, in one embodiment the invention provides a method for the treatment or amelioration of a CD30⁺ proliferative disease or a tumorous disease, the method comprising the step of administering to a subject in need thereof an antibody construct produced according to a method of the invention.

It is preferred that said tumorous disease is a malignant disease, preferably cancer.

In one embodiment of said method for the treatment or amelioration of a disease said malignant disease is selected from the group consisting of Hodgkin lymphoma, Non-Hodgkin lymphoma, leukemia, multiple myeloma and solid tumors.

The following examples further demonstrate the invention and are not intended to be limiting.

### Example 1

### Chromatography

The example describes the purification of a CD30×CD16A bispecific antibody (SEQ ID NO:13) using HCIC chromatography according to the present invention.

The CD30×CD16A bispecific tandem diabody having the amino acid sequence as depicted in SEQ ID NO:13 was produced in Chinese hamster ovary (CHO) cells as previously described (Reusch, U. et al., mAbs 6:3, 727-738, 2014). The protein concentration in the cell culture supernatent was 13 mg/L.

HCIC chromatography was carried out using MEP HyperCell™ resin for capture. The load of the column was calculated as shown in Table 1. The variation of the concentration of the load was as follows:

| | |
|---|---|
| 0: MEP eluate | 0.4 mg/mL |
| +: concentrated MEP eluate | 0.8 mg/mL |
| −: diluted MEP eluate | 0.2 mg/mL |

The CD30×CD16A fraction was diluted at pH 3.7-4.0 as shown in Table 2.

TABLE 1
Yield calculation of the acidic eluate of MEP-chromatography
(determination of concentration by API-ELISA)

| | Volume | Concentration | Amount | Yield |
|---|---|---|---|---|
| Fraction | [mL] | [mg mL⁻¹] | [mg] | [%] |
| Load | 8483.0 | 0.013 | 110.3 | 100 |
| MEP Eluate | 395.6 | 0.280 | 110.8 | 100 |

### Kinetics

The pH of the acidic eluates was raised to pH 7.0 with Tris_054_0.5M_pH10.5) at 0h (T0), 4h (T1), 18h (T2), 24h (T3) and 48h (T4).

Concentration of the protein was measured by apoptosis inhibitor (API) ELISA with 0.5 mL samples.

TABLE 2
Yield based on the acidic eluate in % (API-ELISA)

| | Temperature | | | | T1 | T2 | T3 | T4 |
|---|---|---|---|---|---|---|---|---|
| Sample | (° C.) | Concentration | pH | To | 4 h | 18 h | 24 h | 48 h |
| A1 | 5 | O | 3.7 | 74 | 98 | 141 | 151 | 153 |
| A1 | −70 | O | 3.7 | 72 | 97 | 136 | 149 | 150 |
| A2 | 5 | O | 3.7 | 77 | 88 | 140 | 149 | 163 |
| A3 | 5 | O | 3.7 | 71 | 88 | 126 | 157 | 140 |
| B | 5 | + | 4.0 | 61 | 65 | 99 | 103 | 126 |
| C | 5 | + | 3.4 | 64 | 117 | 55 | 41 | 20 |
| D | 5 | − | 4.0 | 67 | 68 | 67 | 73 | 110 |
| E | 5 | − | 3.4 | 99 | 129 | 94 | 85 | 70 |
| F | 15 | O | 3.7 | 72 | 97 | 103 | 123 | 92 |
| G | 25 | O | 3.7 | 77 | 82 | 20 | 11 | 11 |

O: Eluate 0.294 mg/mL
+: Concentrated eluate (factor 2) 0.588 mg/mL
−: Diluted eluate (factor 2) 0.147 mg/mL

Samples A1, A2 and A3 show a yield of above 150% after 48h. Storing of the samples at pH 7.0 at −70° C. (probe A4, not shown) did not significantly influence the measuring of concentration by API-ELISA.

Variation of pH and concentration does not influence the measuring of concentration. A higher pH (B, D) shows a lower reactivity kinetic wherein this kinetic is lower at lower concentrations. The low yield is not caused by a fragmentation of the samples. A low pH (C, E) results in a lower yield caused by fragmentation. A higher concentration appears to increase this effect. However, the values after 4h may assume that a lower pH value increases the reactivation.

The temperature shows the effect that a fragmentation by proteolytic activity is strongly increased at higher temperatures. Storing at 15° C. (F) for 18h shows compared to 5° C. (A1, A2, A3) a lower yield due to fragmentation.

### SDS-PAGE Analysis

The neutralized eluates have not significantly changed over the time. Only the acidic eluate stored at 2-8° C. showed a fragmentation which was not observed with the probe stored at −70° C.

A low pH results in a reduction of intact monomers at 50 kDa and additional bands.

Increase of temperature to 25° C. results after 48h in a banding pattern without an intact molecule of four domains.

### Isoelectric Focusing (IEF)-Analysis

The IEF analysis correlated with the banding pattern of the SDS-PAGE analysis. Samples with fragmentation also showed a different pattern of isoforms.

### Size Exclusion (SE)-HPLC Analysis

The amount of monomers increased during the incubation from 75% to 91% and correlated with the respective yields. The samples show 91% monomer after 48h independent from the storage of the samples. The acidic eluate of A1 (−70° C.) could not be assessed, because no UV-signal was detected.

The values of the monomers after 48h correlated with the yields. Samples with varying pH and concentration showed lower yields (F, G) and high amounts of monomers (Table 3).

TABLE 3
Amount of monomer in %

| | Temperature | | | | | T1 | T2 | T3 | T4 |
|---|---|---|---|---|---|---|---|---|---|
| Sample | (° C.) | Concentration | pH | SE | T0 | 4 h | 18 h | 24 h | 48 h |
| A1 | 5 | O | 3.7 | 94 | 76 | 80 | 88 | 89 | 91 |
| A1 | −70 | O | 3.7 | n.a. | 77 | 81 | 88 | 89 | 91 |
| A2 | 5 | O | 3.7 | n.t. | n.t. | n.t. | n.t. | n.t. | 91 |
| A3 | 5 | O | 3.7 | n.t. | n.t. | n.t. | n.t. | n.t. | 91 |
| B | 5 | + | 4.0 | n.t. | n.t. | n.t. | n.t. | n.t. | 78 |
| C | 5 | + | 3.4 | n.t. | n.t. | n.t. | n.t. | n.t. | 77 |
| D | 5 | − | 4.0 | n.t. | n.t. | n.t. | n.t. | n.t. | 89 |
| E | 5 | − | 3.4 | n.t. | n.t. | n.t. | n.t. | n.t. | 86 |
| F | 15 | O | 3.7 | n.t. | n.t. | n.t. | n.t. | n.t. | 92 |
| G | 25 | O | 3.7 | n.t. | n.t. | n.t. | n.t. | n.t. | 96 |

O: Eluate 0.294 mg/mL
+: Concentrated eluate 0.588 mg/mL
−: Diluted eluate 0.147 mg/mL

The calculation of the area relating to the theoretical amounts of protein loaded in the column demonstrated differences. Based on the values of the samples, values below 4.5 indicate loss of protein due to precipitation. This is observed with all MEP-eluates which show a fragmentation due to low-pH or increased temperature.

TABLE 4
SE-HPLC data

| | | | | Theoretical | Theoretical | | |
|---|---|---|---|---|---|---|---|
| | Temperature | | | Concentration | Amount of | Area | Quotient (AU × |
| Sample | (° C.) | Concentration | pH | (mg/mL) | Protein (mg) | (AU × s) | s/mg)/1000 |
| A1 | 5 | O | 3.7 | 0.108 | 0.022 | 95.7 | 4.4 |
| A2 | 5 | O | 3.7 | 0.108 | 0.022 | 95.2 | 4.4 |
| A3 | 5 | O | 3.7 | 0.108 | 0.022 | 94.8 | 4.4 |
| B | 5 | + | 4.0 | 0.240 | 0.048 | 227.5 | 4.7 |
| C | 5 | + | 3.4 | 0.122 | 0.024 | 25.1 | 1.0 |
| D | 5 | − | 4.0 | 0.060 | 0.012 | 56.2 | 4.7 |
| E | 5 | − | 3.4 | 0.035 | 0.007 | 18.4 | 2.6 |
| F | 15 | O | 3.7 | 0.108 | 0.022 | 72.3 | 3.3 |
| G | 25 | O | 3.7 | 0.108 | 0.022 | 22.5 | 1.0 |
| H | 5 | O | 3.7 | 0.194 | 0.039 | 58.5 | 1.5 |
| I | 25 | O | 3.7 | 0.194 | 0.039 | 59.2 | 1.5 |

### Discussion

MEP-chromatography showed a yield of 100% based on the acidic eluate measured in API-ELISA. The three samples at standardized conditions showed a yield of 150% after 48h. The increase in activity correlated with the amount of monomer which increased to 90% till the end. In addition, no significant fragmentations were observed. Storage at −70° C. appears to be possible for the neutralized eluates.

Increase of temperature to 25° C. resulted in a decrease of activity due to fragmentation.

Variation of pH and concentration influences the yield as well as fragmentation. A low pH resulted in a fragmentation of the monomer. However, the banding pattern was different compared to the samples of the temperature study. The banding pattern showed additional and more intense bands at 35, 25 and 12 kDa, but also showed significantly slurred samples. In contrast, a higher pH did not result in a fragmentation but the increase of yield over time was slowed down as well as the yields after 48h (110% and 126%).

A higher concentration at higher pH causes a faster kinetic of reactivation and an increased fragmentation at a lower pH.

SDS-PAGE and IEF analysis showed the correlation between loss of yield due to fragmentation, but does not illustrate the kinetics of reactivation. In SE-HPLC the loss of yield correlated until no fragmentation has been initiated. After fragmentation has started protein is lost due to precipitation which distorts the SE-HPLC result false positive.

The results show that two different and relating to activity counteracting processes are running during the acidic incubation. First, the molecules are reactivated and stabilized which relates to aggregation. Second, a proteolytic process is running at acidic pH which is sensitive to pH and concentration.

### Example 2

### Protein L Chromatography

This example describes the purification of a CD30×CD16A bispecific antibody (SEQ ID NO:13) using protein L chromatography according to the invention.

The CD30×CD16A bispecific antibody (SEQ ID NO:13) was produced in Chinese hamster ovary (CHO) cells as previously described (Reusch, U. t al., mAbs 6:3, 727-738, 2014). Prior to application onto the protein L column the cell-free harvest (ZA) is filtered using a 0.2 μm clear filter.

Protein L chromatography was carried out using Toyopearl AF-rProtein L-650F resin from Tosoh for capture. Alternatively, Capto L material from GE may be used as resin for the capture step. The loading was carried out with a residence time of 4 minutes and the CD30×CD16A bispecific antibody bound specifically to the resin. By a subsequent washing step, non-specifically bound substances were removed and the CD30×CD16A bispecific antibody was then eluted with an acidic buffer (50 mM acetic acid (HOAc/NaOAc), pH 3.3). The acidic pH-value of the eluate then served as incubation step at low pH. The pH of the eluate is at pH 3.4 to 3.6 and the protein L eluate is incubated at room temperature for 48 to 96 hours. Besides inactivation of potential viruses, it was observed that incubation at low pH, preferably at room temperature, led to an increase of active CD30×CD16A bispecific antibody recovery (“product activation”). Further reduction of high molecular weight (HMW) forms was observed. Subsequently, the eluate may be stored at 2° C. to 8° C. without neutralization, for example for a hold time of 5 weeks.

### Product Activation at pH 3.6

The hold time of the eluate at a low pH led to a product activation, which could be detected by binding-ELISA. In contrast to UV-analysis, only active product molecules can be detected. For determination of product activation, the amount of product before and after the hold time was measured by binding-ELISA.

The following formula was used:

Product activation [%]=(amount of product in eluate after hold time [mg]/amount of product in eluate before hold time [mg])×100% In a first test run, incubation was studied at room temperature, 2 to 8° C. and −70° C. for 39 hours (Table 5). The highest product activation of 174% was observed at room temperature. This was further tested in following experiments. Different durations at different temperatures were investigated (Table 6). Hold time at −70° C. already led to notable low product concentrations after 48 hours. The best results were obtained at room temperature (RT) for 48 hours.

TABLE 5
Results of product concentration (ELISA) after hold
time at different temperatures (Experiment A).

| | Conc. |
|---|---|
| Hold time | (ELISA) |
| conditions | [mg/mL] |
| No storage | 1.04 |
| RT for 39 h | 1.81 |
| 2-8° C. for 39 h | 1.60 |
| −70° C. for 39 h | 1.01 |

TABLE 6
Results of product concentration (ELISA) after different
hold times at different temperatures (Experiment B).

| Hold time | RT | 2-8° C. | −70° C. |
|---|---|---|---|
| conditions | Conc. (ELISA) [μg/mL] | | |
| No storage | 500.96 | | |
| 24 h | 573.74 | 554.16 | nyd |
| 48 h | 579.04 | 481.79 | 32.97 |
| 72 h | 530.25 | 542.96 | 21.95 |

In summary, product activation between 116% and 174% within 48 hours at room temperature were observed.

TABLE 7
Results of product activation after 48 hours at RT

| | | Product |
|---|---|---|
| Experiment | Short description | activation [%] |
| A | Hold time of the eluate (pH | 174 |
| | 3.6) at RT, 2-8° C. and −70° C. | |
| | for 39 hours. | |
| B | Hold time of the eluate (pH | 116 |
| | 3.6) at RT for 24, 48 and 72 | |
| | hours. | |
| C | Hold time of the eluate (pH | 148 |
| | 3.6) at RT for 48 hours. | |
| D | Hold time of the eluate (pH | 139 |
| | 3.6) at RT for 48 hours. | |

In the course of further purifications these activation levels were also investigated. The hold time at RT was either 25 or 49 hours. A hold time of 25 hours showed yields of 109 and 126%, a hold time of 49 hours showed 160% (capture CO2). The analysis after product activation was carried out after additional 3 days of hold time at 2 to 8° C. at pH 3.6 or even after longer hold time of 10 days at 2 to 8° C. Product activation before further processing was between 130 and 209% due to the hold time at 2 to 8° C. Consequently, the yields in relation to the amount of product in the cell-free harvest were 163% to 227%.

Since the hold time at pH 3.6 showed a positive effect on the product concentration, a period of 48 to 96 hours at room temperature was determined. Here, the average value of product activation was 131%. The storage of the protein L eluate at pH 3.6 at 2 to 8° C. partially showed a further increase of the product concentration measured by binding-ELISA.

### Reduction of HMW Forms at pH 3.6

In addition to product activation, the formation of high molecular weight (HMW) forms was investigated at different temperatures for the hold time (Table 8). It could be observed that the content of HMW forms increased at −70° C. This is most likely due to the freeze-thaw cycle. Room temperature proved to be the optimal temperature for storage of the protein L eluate. In comparison to the sample without hold time at pH 3.6 a reduction of HMW forms could be observed. This correlates with the product concentrations measured by binding-ELISA, which means that reduction of HMW forms lead to an increase in product activity. It is assumed that the low pH-value destabilizes the HMW forms.

TABLE 8
Results of HMW and LMW forms after hold time at
different hold time conditions (Experiment A).

| | Conc. | HMW | LMW |
|---|---|---|---|
| Hold time | (ELISA) | (SE-HPLC) | (SE-HPLC) |
| conditions | [mg/mL] | [%] | [%] |
| No hold time | 1.04 | 16.50 | 0.38 |
| RT for 39 h | 1.81 | 6.65 | 0.28 |
| 2-8° C. for 39 h | 1.60 | 14.28 | 0.36 |
| −70° C. for 39 h | 1.01 | 23.86 | 0.39 |

Further different hold times were tested (Table 9) to examine the kinetics of the HMW reduction. The sample without hold time revealed 15% of HMW forms and 3.2% of low molecular weight (LMW) forms. Storage at RT as well as 2 to 8° C. led to a reduction of HMW and LMW forms. A relation between product concentration by binding-ELISA and the level of HMW forms could be observed. Samples with lower levels of HMW forms tend to have higher product concentration, measured by binding-ELISA, meaning that a higher level of active product is present.

In addition, storage at 2 to 8° C. after the optimal hold time of 48 hours at RT was examined (Tables 10 and 11). Further reduction of HMW and LMW forms could be observed.

It could be shown that the hold time at pH 3.6 is also beneficial for reduction of HMW forms. The reduction of HMW forms also appeared to be linked to the product concentration measured by binding-ELISA. An increase of the product concentration was noted when HMW forms were reduced.

TABLE 9
Content of HMW and LMW forms after hold time at
pH 3.6 at different hold time conditions.

| | RT | | | 2-8° C. | | | −70° C. | | |
|---|---|---|---|---|---|---|---|---|---|
| | Conc. | | | Conc. | | | Conc. | | |
| Hold time | (ELISA) | HMW | LMW | (ELISA) | HMW | LMW | (ELISA) | HMW | LMW |
| conditions | [mg/mL] | [%] | [%] | [mg/mL] | [%] | [%] | [mg/mL] | [%] | [%] |
| 24 h | 573.74 | 5.70 | 2.60 | 554.16 | 10.31 | 2.59 | / | 23.03 | 6.36 |
| 48 h | 579.04 | 0.05 | 0.79 | 481.79 | 6.80 | 0.70 | 32.97 | 16.23 | 0.88 |
| 72 h | 530.25 | 3.41 | 1.19 | 542.96 | 6.32 | 0.51 | 21.95 | 24.62 | 1.11 |

TABLE 10
Results after storage for 1 and 2 weeks at 2-8° C.

| | HMW | LMW |
|---|---|---|
| | (SE-HPLC) | (SE-HPLC) |
| Hold time and storage conditions | [%] | [%] |
| No hold time | 8.27 | 0.95 |
| RT for 48 h | 5.19 | 0.90 |
| RT for 48 h, then 1 week at 2-8° C. | 4.22 | 0.28 |
| RT for 48 h, then 2 week at 2-8° C. | 1.99 | 0.43 |

TABLE 11
Results after storage for 1 and 2 weeks at 2-8° C.

| | HMW | LMW |
|---|---|---|
| | (SE-HPLC) | (SE-HPLC) |
| Hold time and storage conditions | [%] | [%] |
| RT for 96 h, then 1 week at 2-8° C. | 2.24 | 0.04 |
| RT for 96 h, then 2 weeks at 2-8° C. | 1.62 | 0.00 |
| 2-8° C. for 96 h, then 1 week at 2-8° C. | 1.59 | 0.07 |
| 2-8° C. for 96 h, then 2 weeks at 2-8° C. | 1.59 | 0.04 |

### Hold Time of Low pH Incubation Step at 2 to 8° C.

For investigations of the hold time of the low pH incubation step at 2 to 8° C. pH adjustment of the protein L eluate to pH 5.0 and 7.0 was conducted. Either the protein L eluate was first incubated at pH 3.6 at RT for 48 hours or directly adjusted to a pH-value of 5.0 or 7.0. Re-measurements were done after one and two weeks of storage at 2 to 8° C. Results are summarized in Table 12.

Comparison between sample 1 and 2 confirmed that hold time at pH 3.6 leads to product activation and reduction of HMW forms. The samples incubated at pH 3.6 at RT for 48 hours and then neutralized to pH 5.0 showed a clear trend towards lower levels of HMW forms.

TABLE 12
Results of hold time of VINI at 2-8° C. (V12).

| | | | Conc. | HMW | LMW |
|---|---|---|---|---|---|
| Sample | | Conc. (UV) | (ELISA) | (SE-HPLC) | (SE-HPLC) |
| No. | Hold time conditions | [mg/mL] | [mg/mL] | [%] | [%] |
| 1 | pH to 5.0 | 1.15 | 0.39 | 33.00 | 0.60 |
| 2 | RT for 48 h at pH | 1.16 | 0.64 | 10.58 | 0.59 |
| | 3.6, then pH 5.0 | | | | |
| 3 | RT for 48 h at pH 3.6, | 1.11 | 0.65 | 21.42 | 0.19 |
| | then pH 5.0 & 1 week at 2-8° C. | | | | |
| 4 | RT for 48 h at pH 3.6, | 1.20 | 0.65 | 33.36 | 0.24 |
| | then pH 5.0 & 2 weeks at 2-8° C. | | | | |
| 5 | RT for 48 h at pH 3.6, then pH | 1.13 | 0.59 | 17.05 | 0.46 |
| | 7.0 | | | | |
| 6 | RT for 48 h at pH 3.6, | 0.72 | 0.72 | 13.86 | 0.14 |
| | then pH 7.0 & 1 week at 2-8° C. | | | | |
| 7 | RT for 48 h at pH 3.6, | 0.55 | 0.55 | 11.53 | 0.29 |
| | then pH 7.0 & 2 weeks at 2-8° C. | | | | |

Product concentrations, measured by UV and binding-ELISA, stayed nearly constant over the hold time at 2 to 8° C. But the level of HMW forms increased from 11% to 33%. For pH 7.0, the content of HMW forms was even higher with 17% in the beginning but decreased over time of storage. Simultaneously, the product concentration decreased to 0.6 g/L after 2 weeks. This might be due to formation of precipitates which might be still measurable by binding-ELISA.

In conclusion, hold time of neutralized protein L eluate leads to further formation of HMW forms. For following polishing steps the pH needs to be adjusted just before application to reduce the hold time at higher pH to a minimum. Filtration might be necessary prior to the polishing step as turbidity could appear.

### CONCLUSIONS

It is assumed that the product is present in both active as well as non-active conformations in the cell-free harvest. Via protein L affinity chromatography both variants of the product bind specifically to the resin. During the following washing step, non-specifically bound contaminants are removed. Subsequently, the product is eluated due to a decrease of pH using an acidic buffer. There is a correlation between the product concentration in the protein L eluate and the formation of HMW forms. Higher product concentrations resulted in higher levels of HMW forms. It was observed that the following low pH treatment at 3.6 leads to a reduction of HMW forms as well as an increase of product concentration measured by binding-ELISA. Contrary to UV-analysis, which measures the total amount of product in the sample, the binding-ELISA measurement only detects the product in its active conformation or at least in a conformation that enables binding to the antigen. It is assumed that the incubation at low pH facilitates conformational change of the initially non-active forms into active conformations. Presumably, the CD30×CD16A antibody tertiary and/or quaternary structure is destabilized at low pH and, thus, can relocate its subunits under these conditions. Consequently, the concentration measured by binding-ELISA increases during the low pH hold step while the concentration measured by UV remains unchanged.

### Example 3

### Further Purification and Polishing Steps

After the chromatography for capture and incubating the antibody construct at low pH further steps can be chosen for the purification process. An example for a purification process of the CD30×CD16A bispecific antibody is described in the following:

The incubation step at low pH is followed by a concentration and first diafiltration step into buffer suitable for polishing. Polishing for depletion of contaminants consists of an anion exchange chromatography (AEX) run in flow-through mode and a hydroxyapatite chromatography (HAC) run in bind-and-elute mode. Subsequently, virus filtration is conducted. Finally, the concentration and second diafiltration step into buffer suitable for formulation is performed.

Sequence table:

| SEQ | | |
|---|---|---|
| ID NO | Description | Sequence |
| 1. | CD16 | SYYMH |
| | CDR H1 | |
| 2. | CD16 | IINPSGGSTSYAQKFQG |
| | CDR H2 | |
| 3. | CD16 | GSAYYYDFADY |
| | CDR H3 | |
| 4. | CD16 | GGHNIGSKNVH |
| | CDR L1 | |
| 5. | CD16 | QDNKRPS |
| | CDR L1 | |
| 6. | CD16 | QVWDNYSVL |
| | CDR L1 | |
| 7. | CD30 | TYTIH |
| | CDR H1 | |
| 8. | CD30 | YINPSSGYSDYNQNFKG |
| | CDR H2 | |
| 9. | CD30 | RADYGNYEYTWFAY |
| | CDR H3 | |
| 10. | CD30 | KASQNVGTNVA |
| | CDR L1 | |
| 11. | CD30 | SASYRYS |
| | CDR L1 | |
| 12. | CD30 | QQYHTYPLT |
| | CDR L1 | |
| 13. | CD16x CD30 | QVQLVQSGAEVKKPGESLKVSCKASGYTFTSYYMHWVRQAPGQGLEWMGIINPSGGS |
| | bispecific Ab | TSYAQKFQGRVTMTRDTSTSTVYMELSSLRSEDTAVYYCARGSAYYYDFADYWGQGT |
| | construct | LVTVSSGGSGGSGGSDIVMTQSPKFMSTSVGDRVTVTCKASQNVGTNVAWFQQKPGQ |
| | | SPKVLIYSASYRYSGVPDRFTGSGSGTDFTLTISNVQSEDLAEYFCQQYHTYPLTFG |
| | | GGTKLEINGGSGGSGGSQVQLQQSGAELARPGASVKMSCKASGYTFTTYTIHWVRQR |
| | | PGHDLEWIGYINPSSGYSDYNQNFKGKTTLTADKSSNTAYMQLNSLTSEDSAVYYCA |
| | | RRADYGNYEYTWFAYWGQGTTVTVSSGGSGGSGGSSYVLTQPSSVSVAPGQTATISC |
| | | GGHNIGSKNVHWYQQRPGQSPVLVIYQDNKRPSGIPERFSGSNSGNTATLTISGTQA |
| | | MDEADYYCQVWDNYSVLFGGGTKLTVL |
| 14. | VH CD16 | QVQLVQSGAEVKKPGESLKVSCKASGYTFTSYYMHWVRQAPGQGLEWMGIINPSGGS |
| | | TSYAQKFQGRVTMTRDTSTSTVYMELSSLRSEDTAVYYCARGSAYYYDFADYWGQGT |
| | | LVTVSS |
| 15. | VL CD16 | SYVLTQPSSVSVAPGQTATISCGGHNIGSKNVHWYQQRPGQSPVLVIYQDNKRPSGI |
| | | PERFSGSNSGNTATLTISGTQAMDEADYYCQVWDNYSVLFGGGTKLTVL |
| 16. | VH CD30 | SYVLTQPSSVSVAPGQTATISCGGHNIGSKNVHWYQQRPGQSPVLVIYQDNKRPSGI |
| | | PERFSGSNSGNTATLTISGTQAMDEADYYCQVWDNYSVLFGGGTKLTVL |
| 17. | VL CD30 | DIVMTQSPKFMSTSVGDRVTVTCKASQNVGTNVAWFQQKPGQSPKVLIYSASYRYSG |
| | | VPDRFTGSGSGTDFTLTISNVQSEDLAEYFCQQYHTYPLTFGGGTKLEIN |
| 18. | C-terminal | SFFPPGYQ |
| | CD16a | |
| 19. | Human | GMRTEDLPKAVVFLEPQWYRVLEKDSVTLKCQGAYSPEDNSTQWFHNESLISSQASS |
| | CD16A | YFIDAATVDDSGEYRCQTNLSTLSDPVQLEVHIGWLLLQAPRWVFKEEDPIHLRCHS |
| | | WKNTALHKVTYLQNGKGRKYFHHNSDFYIPKATLKDSGSYFCRGLFGSKNVSSETVN |
| | | ITITQGLAVSTISSFFPPGYQ |
| 20. | hexa- | HHHHHH |
| | histidine tag | |
| 21. | L1 | GGSGGSGGS |
| 22. | L2 | GGSGGSGGS |
| 23. | L3 | GGSGGSGGS |

1. A method for the production of bispecific antibody construct comprising a first binding domain for FcγRIII and a second binding domain for CD30, the method comprising the following steps
(a) chromatographically capturing the antibody construct from a solution;
(b) eluting the antibody construct from the capture matrix;
(c) reducing the pH in the solution of the eluted antibody construct to low pH in a range of 2.5 pH to 3.9 pH and incubating the antibody construct under these conditions for at least 40h;
(d) neutralizing to a pH in the range of pH 4.5 to pH 8.0.

2. The method according to claim 1, wherein the first binding domain of the antibody construct for FcγRIII binds to CD16A.

3. The method according to claim 2, wherein the antibody construct comprises at least four variable domains from the group consisting of:
(a) heavy chain variable domain specific for CD16A (VH_CD16A) comprising a heavy chain CDR1 having the amino acid sequence set forth in SEQ ID NO:1, a heavy chain CDR2 having the amino acid sequence set forth in SEQ ID NO:2, a heavy chain CDR3 having the amino acid sequence set forth in SEQ ID NO: 3;
(b) a light chain variable domain specific for CD16A (VL_CD16A) comprising a light chain CDR1 having an amino acid sequence set forth in SEQ ID NO:4, a light chain CDR2 having an amino acid sequence set forth in SEQ ID NO: 5, and a light chain CDR3 having an amino acid sequence set forth in SEQ ID NO: 6;
(c) heavy chain variable domain specific for CD30 (VH_CD30A) comprising a heavy chain CDR1 having the amino acid sequence set forth in SEQ ID NO:7, a heavy chain CDR2 having the amino acid sequence set forth in SEQ ID NO:8, a heavy chain CDR3 having the amino acid sequence set forth in SEQ ID NO: 9;
(d) a light chain variable domain specific for CD30A (VL_CD30A) comprising a light chain CDR1 having an amino acid sequence set forth in SEQ ID NO:10, a light chain CDR2 having an amino acid sequence set forth in SEQ ID NO: 11, and a light chain CDR3 having an amino acid sequence set forth in SEQ ID NO: 12.

4. The method according to claim 1, wherein the variable domains of the antibody construct are linked one after another by peptide linkers L1, L2 and L3 consisting of 12 or less amino acid residues and positioned within each of the two polypeptide chains from the N-terminus to the C-terminus in the order: VH_CD30-L1-VL_CD16A-L2-VH_CD16A-L3-VL_CD30.

5. The method according to claim 3, wherein linker L2 of the antibody construct consists of 3 to 9 amino acid residues.

6. The method according to claim 1, wherein the antibody construct comprises an amino acid sequence as set forth in SEQ ID NO:13.

7. The method according to claim 1, wherein the pH in step (c) is in the range of pH 3.0 to pH 3.75.

8. The method according to claim 1, wherein the antibody construct is incubated in step (c) for at least 48h.

9. The method according to claim 1, wherein the chromatographic capturing method in step (a) is selected form the group consisting of a protein L chromatography, an anion exchange chromatography (AEX), a cation exchange chromatography (CEX), a hydrophobic interaction chromatography (HIC) or a mixed mode chromatography (MMC).

10. The method according to claim 1, wherein the method further comprises the additional steps:
(e) capturing the antibody construct from the solution by at least one chromatographic method selected from the group consisting of an anion exchange chromatography (AEX), a cation exchange chromatography (CEX), a hydrophobic interaction chromatography (HIC) or a mixed mode chromatography (MMC).

11. The method according to claim 1, wherein the elution of the antibody construct in step (b) is performed using a buffer selected from the group consisting of buffers comprising sodium acetate/acetic acid, sodium formiate/formic acid, sodium citrate/citric acid, and sodium succinate/succinic acid.

12. The method according to claim 1, wherein the antibody construct is formulated as a pharmaceutical composition in a step (f).

13. An antibody construct produced by the method according to claim 1.

14. A pharmaceutical composition comprising an antibody construct produced by the method according to claim 1 for the treatment or amelioration of a CD30⁺ proliferative disease or a tumorous disease.

15. A method for the treatment or amelioration of a patient, the method comprising administering to a subject suffers from a CD30⁺ proliferative disease or a tumorous disease an antibody construct produced by a method according to claim 1.


SEQUENCE LISTING (21 sequences)

SEQ ID NO: 1 - protein, 5 aa, Artificial Sequence
  CDRH1
SYYMH

SEQ ID NO: 2 - protein, 17 aa, Artificial Sequence
  CDR H2
IINPSGGSTSYAQKFQG

SEQ ID NO: 3 - protein, 11 aa, Artificial Sequence
  CDR H3
GSAYYYDFADY

SEQ ID NO: 4 - protein, 11 aa, Artificial Sequence
  CDR L1
GGHNIGSKNVH

SEQ ID NO: 5 - protein, 9 aa, Artificial Sequence
  CDR L2
QVWDNYSVL

SEQ ID NO: 6 - protein, 9 aa, Artificial Sequence
  CDR L3
QVWDNYSVL

SEQ ID NO: 7 - protein, 5 aa, Artificial Sequence
  CDR H1
TYTIH

SEQ ID NO: 8 - protein, 17 aa, Artificial Sequence
  CDR H2
YINPSSGYSDYNQNFKG

SEQ ID NO: 9 - protein, 14 aa, Artificial Sequence
  CDR H3
RADYGNYEYTWFAY

SEQ ID NO: 10 - protein, 11 aa, Artificial Sequence
  CDR L1
KASQNVGTNVA

SEQ ID NO: 11 - protein, 7 aa, Artificial Sequence
  CDR L2
SASYRYS

SEQ ID NO: 12 - protein, 9 aa, Artificial Sequence
  CDR L3
QQYHTYPLT

SEQ ID NO: 13 - protein, 483 aa, Artificial Sequence
  CD16xCD30
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGSGGSGGSDIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEINGGSGGSGGSQVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 14 - protein, 120 aa, Artificial Sequence
  VH CD16
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 15 - protein, 106 aa, Artificial Sequence
  VL CD16
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 16 - protein, 106 aa, Artificial Sequence; identical to SEQ ID NO: 15
  VH CD30

SEQ ID NO: 17 - protein, 107 aa, Artificial Sequence
  VL CD30
DIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEIN

SEQ ID NO: 18 - protein, 8 aa, Artificial Sequence
  C-terminal CD16A
SFFPPGYQ

SEQ ID NO: 19 - protein, 192 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 20 - protein, 6 aa, Artificial Sequence
  His tag
HHHHHH

SEQ ID NO: 21 - protein, 9 aa, Artificial Sequence
  linker
GGSGGSGGS